(19) United States
(12) Patent Application Publication
(10) Pub. No.: US 2013/0065792 A1
(43) Pub. Date: Mar. 14, 2013

(54) INDUCIBLE EXPRESSION OF SGLT5, METHODS AND KITS USING THE SAME

(71) Applicants: Peter EICKELMANN, Mittelbiberach (DE); Stefanie FROEHNER, Muenchen (DE); Rolf GREMPLER, Birkenhard/Warthausen (DE); Michael MARK, Biberach an der Riss (DE)

(72) Inventors: Peter EICKELMANN, Mittelbiberach (DE); Stefanie FROEHNER, Muenchen (DE); Rolf GREMPLER, Birkenhard/Warthausen (DE); Michael MARK, Biberach an der Riss (DE)

(73) Assignee: BOEHRINGER INGELHEIM INTERNATIONAL GMBH, INGELHEIM AM RHEIN (DE)

(21) Appl. No.: 13/419,784

(22) Filed: Mar. 14, 2012

(30) Foreign Application Priority Data
Mar. 16, 2011 (EP) 11158390.2

(51) Int. Cl. C12N 15/12; C12N 15/63; C12N 1/21; C12Q 1/54; C12N 5/10; C12N 15/79; C12Q 1/02; C40B 30/06; C12N 15/11; C12N 1/19

(52) U.S. Cl. 506/10; 536/23.1; 536/23.5; 435/320.1; 435/252.3; 435/254.2; 435/348; 435/325; 435/365; 435/364; 435/350; 435/369; 435/367; 435/358; 435/455; 435/471; 435/29; 435/14

(57) ABSTRACT

The present invention pertains to the function of SGLT5 as a sodium dependent co-transporter of mannose and/or fructose. The invention provides nucleic acids and host cells for inducible expression of SGLT5. Based on an inducible expression system the invention provides methods, assays and test kits for the identification of compounds interacting with SGLT5, especially with human SGLT5.

### FIELD OF THE INVENTION

The present invention elucidates the function of SGLT5, and provides tools to further study SGLT5 as well as methods, kits and articles of manufacture related to SGLT5 and its function. The present inventors have established that SGLT5 acts as a sodium dependent mannose and/or fructose cotransporter. The inventors have further established that SGLT5 is advantageously expressed in an inducible manner in host cells. Constitutive expression prevents stable and reproducible transporter function. Based on these findings the invention provides test methods, assays, kits and articles of manufacture. For example, assays to measure the transport function of SGLT5, optionally in the presence of further compounds, such as small molecule compounds, screening assays to find agonists and/or antagonists of SGLT5 and assays to find further substrates of SGLT5 are provided.

### BACKGROUND ART

The so-called SGLT family comprises a large number of proteins, with estimates exceeding 220 members. The family is named after the prototypic member, the Na+/glucose cotransporter, SGLT1. The various family members share certain structural features with SGLT1 (Turk and Wright, 1996). The family includes sequences from a wide variety of organisms, such as mammalian, eubacterial, yeast, insect and nematode sequences. In humans, 11 different SGLT genes (slc5a genes) are currently known and are expressed in a diverse range of tissues, from epithelial cells to the CNS (Wright and Turk, 2004, Turk et al., 1996; Jung, 2002)

It has previously been discussed that various members of the SGLT family share a similar or identical topology of membrane spanning elements. Turk and Wright, 1996, have proposed a common core of 13 transmembrane helices across the family. Some members, like SGLT1 itself, have one additional span appended to the C-terminus, and still others, two.

The function of some members of the family, most notably of the prototypic Na+/glucose cotransporter SGLT1 has been elucidated. For other members of the family little is known about their function. Cotransport of a variety of substrates such as sugars, choline, multiple vitamins, inositol, proline, pantothenate, iodide, urea and undetermined solutes has been suggested (Turk and Wright, 1996).

One particular member of the SGLT family, SGLT5, has been identified in rabbit (GenBank: U08813, 1959 bp, mRNA, Oryctolagus cuniculus Na+/glucose cotransporter-related protein, complete cds), human (GenBank: AK057946, 2140 bp; mRNA, Homo sapiens cDNA FLJ25217 f is, clone REC08938, highly similar to Oryctolagus cuniculus Na+/glucose cotransporter-related protein), rat (NCBI Reference Sequence: NM_{—}001107007.1, 1791 bp, mRNA, Rattus norvegicus solute carrier family 5 (sodium/glucose cotransporter), member 10 (Slc5a10)) and bovine (GenBank: AY514442, 2079 bp, mRNA, Bos taurus Na+/glucose cotransporter-related protein mRNA, complete cds). In humans, the gene (slc5a10) is located on chromosome 17 and has 14 exons. The function or physiological role of SGLT-5 is not established. Expression patterns have been reported, with a predominant expression in kidney (Pajor A M., 1994; Leicht S., 2005; Leicht et al., 2006, Zhao et al., 2005a), some expression in e.g. bovine testes, skeletal muscle and spleen (Zhao et al., 2005a) and expression in porcine jejunum, liver, kidney and skeletal muscle (Aschenbach et al., 2009). Moreover, multiple splicing variants of SGLT5 have been reported (Zhao et al., 2005a)

Leicht S., 2005, p. 58 ff, discloses an experiment with constitutive expression of SGLT5 by a HEK/hSGLT5 cell line. Leicht S., 2005, p. 60 ff. further discloses an experiment of tetracycline inducible expression of SGLT5 by a T-REx/hSGLT5 cell line. But these cells failed to show SGLT5 activity (page 62 of Leicht S., 2005).

The present invention aims at elucidating the function of SGLT5 and providing tools for studying this protein.

It was desired to establish a stable cell line expressing SGLT5 to allow an analysis of this transporter, especially to establish an inducible expression of SGLT5.

### SUMMARY OF THE INVENTION

The present inventors have succeeded in providing a test system for investigating the function of SGLT5, in particular human SGLT5 (hSGLT5). It has been found that constitutive expression of SGLT5 according to Leicht S., 2005 or Leicht et al., 2006, leads to unstable and irreproducible assay results. But by the development of an inducible expression system, the current inventors have succeeded in establishing the function of SGLT5 as a sodium dependent mannose and/or fructose co-transporter. Based on this finding, methods, assays and test kits are provided.

More specifically, the present invention relates to a nucleic acid comprising one or more nucleotide sequences for inducible expression of a member of the SGLT co-transporter family having the function of sodium dependent co-transport of mannose and/or fructose, preferably wherein the member of the SGLT co-transporter family transporting mannose and/or fructose is SGLT5, more preferably human SGLT5. In particular embodiments of the invention, the nucleic acid coding for a member of the SGLT co-transporter family having the function of sodium dependent co-transport of mannose and/or fructose is selected from any one of a) to d)

a) a nucleic acid encoding the amino acid sequence according to SEQ ID No. 2;

b) a nucleic acid according to SEQ ID No. 1;

c) a nucleic acid having at least 65% identity to a nucleic acid according to a) or b) and encoding a member of the SGLT co-transporter family having the function of sodium dependent co-transport of mannose and/or fructose;

d) a nucleic acid sequence hybridizing under stringent conditions to the complement of any one of nucleic acid sequences according to a) to c) and encoding a member of the SGLT co-transporter family having the function of sodium dependent co-transport of mannose and/or fructose.

In exemplary, non limiting embodiments of the invention, the nucleic acid as defined above can be selected from a vector, plasmid, YAC, BAC, or recombinant virus.

In particular embodiments the present invention provides nucleic acids which comprise an inducible expression system. Examples for such expression systems are basically known in the art.

Appropriate inducible expression systems are characterized by the following non limiting list of inducers:

a) Tetracycline or tetracycline derivative doxycycline (Tet-on or Tet-off); in such systems expression of the gene of interest is induced by the presence or removal of tetracycline or the tetracycline derivative doxcycline. Examples for a Tet-on system are commercially available, e.g. from the provider Invitrogen (Carlsbad, Calif., USA) in which binding of tetracycline to the tetracycline repressor leads to derepression of the promoter. The provider Clontech Laboratories (Mountain View, Calif., USA) provides both Tet-off and Tet-on systems in which a tetracycline-responsive transcriptional activator binds to a tetracycline-responsive element either in the absence (Tet-off) or presence (Tet-on) of tetracycline or the tetracycline derivative doxycycline and thereby activitates the promoter.

b) Ecdyson or its analog muristerone A; examples comprising activation of a minimal heat shock promoter upon muristerone A induced binding of a heterodimer of the ecdysone receptor and the retinoid X receptor to ecdysone/glucocorticoid response elements are commercially available, e.g. from the provider Invitrogen.

c) Mifepristone; also this system can be purchased e.g. from the provider Invitrogen under the trade name Gene Switch system®.

d) temperature shift acting on a temperature sensitive promoter; in this case which is based on well known bacterial or eukaryotic temperature shock promotors and their natural binding proteins, the activity of the gene can be switched on by a temperature shift which can be a heat shock, e.g. to 42° C. or a cold shock, e.g. to 15° C. or even lower. In another case temperature regulated expression is based on a temperature-regulatable Sindbis virus replicon-based expression system in which expression is induced by temperatures lower than 35° C.

e) transient expression by adenovirus mediated gene transfer; Adenovirus-mediated gene transfer can be used for transient high-level expression in vitro or in vivo. In one preferred embodiment, an additional induction of expression is not needed then, due to the transient nature of adenovirus-mediated expression.

f) inducible expression by retrovirus mediated gene transfer.

The invention furthermore provides host cells comprising at least one nucleic acid as defined herein. The one or more nucleic acids may be integrated into a host cell chromosome or be located extrachromosomally.

In a particular exemplary embodiment, the invention relates to the deposited recombinant host cell having the accession number DSM ACC3096.

Based on the nucleic acids and recombinant cells the present invention also provides assay systems, methods and kits.

Thus, the invention provides a method of preparing cells recombinantly expressing a member of the SGLT co-transporter family having the function of sodium dependent co-transport of mannose and/or fructose comprising the use of a nucleic acid according to any aspect of the present invention.

The invention also provides a method of assessing the transport of a compound, comprising the use of a nucleic acid and/or host cell according to any aspect of the invention, a SGLT co-transporter encoded by a nucleic acid according to any aspect of the invention or produced by a cell according to any aspect of the invention, or a cell obtainable by any aspect of the invention. The invention also provides a method of screening for a compound capable of interacting with a protein encoded by a nucleic acid according to any aspect of the invention, or produced by a cell according to any aspect of the invention. In a further embodiment the invention provides a method for assessing the function (e.g. the function as an agonist or antagonist) of a compound interacting with a protein encoded by a nucleic acid according to any aspect of the invention, or produced by a cell according to any aspect of the invention. Any of the above methods may comprise the use of a host cell according to any of the aspects of the invention or obtainable by any method of the invention.

In more specific embodiments the methods of the present invention may comprise the steps of a) contacting a cell expressing a protein encoded by a nucleic acid according to any aspect of the invention, or a non-cellular system such as a membrane (e.g. a liposome) comprising the protein of the present invention with one or more compounds and b) assessing the transport of a compound, which may be the same or different to the one or more compounds according to step a).

In particular embodiments of the invention the transport assessed is sodium dependent co-transport.

Compounds that can be used in the context of the present invention include

a) small molecular weight chemical entities, preferably with a molecular weight below 500 Da, more preferred between 50 and 250 Da; and

b) sugars, optionally modified by the substitution of a hydroxylgroup by one or more of the chemical groups selected from —H (desoxy), -methyl, -ethyl, -propyl, -isopropyl, -oxomethyl, -oxoethyl, -oxopropyl, -oxoisopropyl, -acetyl, -amino, -imino and/or -acetamido, more in particular monosaccharides or disaccharides, e.g. one or more selected from glucose, fructose, mannose, galactose or alpha-methyl glucopyranoside or imino sugars.

In a preferred embodiment of the invention the compound is mannose and/or fructose.

The invention encompasses competitive methods, kits and articles of manufacture, in particular kits and articles of manufacture for use in and/or suitable for the methods of the present invention. Thus, kits or articles of manufacture of the present invention may comprise one or more selected from at least one nucleic acid according to any aspect of the invention; a protein encoded by the nucleic acid according to any aspect of the invention or produced by a cell according to any aspect of the invention; at least one host cell according to any aspect of the invention, or at least one cell obtainable by the methods of the invention.

### BRIEF DESCRIPTION OF THE FIGURES

FIG. 1A provides the sequence of full length human SGLT5 contained in pT-Rex DEST 30 hSGLT5; FIG. 1B provides the amino acid sequence of human SGLT5 (AK057946)

FIG. 2: Expression of SGLT5 in T-Rex 293-hSGLT5 cells

FIG. 3: Time dependent specific uptake of different monosaccharides by hSGLT5 expressed in T-Rex 293-hSGLT5 cells

FIG. 4: Km-determination for mannose (4A), fructose (4B) and for AMG (4C) by uptake of [¹⁴C]-Mannose (4A), [¹⁴C]-Fructose (4B) and [¹⁴C]-AMG (4C) by T-Rex 293-hSGLT5 cells, respectively

### DETAILED DESCRIPTION

Unless specified otherwise, all terms used in this description have the meaning common in the art.

As used in the context of the present specification, the terms “saccharide”, “sugar” or “sugars” are used interchangeably and generally include sugars that may optionally be modified by the substitution of a hydroxylgroup by one or more of the chemical groups selected from —H (desoxy), -methyl, -ethyl, -propyl, -isopropyl, -oxomethyl, -oxoethyl, -oxopropyl, -oxoisopropyl, -acetyl, -amino, -imino and/or -acetamido, in particular imino sugars. The term in particular relates to monosaccharides or disaccharides (and respective imino sugars), in particular monosaccharides that are structurally related to mannose and/or fructose.

As used in the context of the present specification, the term “small molecular weight entities” generally relates to small molecular weight entities that preferably have a molecular weight below 500 Da, more preferred between 250 and 50 Da; in particular small molecular weight entities known to interact with SGLT family members, more in particular small molecular weight entities that are similar to or identical with small molecular weight entities known to interact with SGLT5 as exemplified in this specification.

Nucleic Acids of the Invention

The present invention relates to nucleic acids characterized by comprising nucleotide sequences encoding a protein having sodium-dependent cotransporter function for mannose and/or fructose which are suitable for inducible expression. On some occasions such nucleic acids are also referred to as “nucleic acids of the invention”. More specifically, the nucleic acid of the present invention comprises at least one sequence coding for a member of the SGLT co-transporter family having the function of sodium dependent co-transport of mannose and/or fructose.

The SGLT co-transporter family is also referred to as sodium:solute symporter family. It is an extended family comprising more than 220 members in different species. It has previously been discussed that various members of the SGLT family share a similar or identical topology of membrane spanning elements. Turk and Wright, 1996, have proposed a common core of 13 transmembrane helices across the family. Some members, like SGLT1 itself, have one additional span appended to the C-terminus, and still others, two. On the basis of the known family members of the SGLT co-transporter family, the skilled person can readily establish whether a further sequence also belongs to this family. The skilled person can derive further information from e.g. Zhao et al., 2005b, who discusses several conserved sodium:solute symporter family signatures that are characteristic for the family. Accordingly, the skilled person is aware of the sodium:solute symporter family signature 1 (NA_SOLUT_SYMP_{—}1; accession no PS00456), the sodium:solute symporter family signature 2 (NA_SOLUT_SYMP_{—}2; accession no PS00457), and the sodium:solute symporter family profile (PS50283). The nucleic acid of the present invention will advantageously be characterized by one or more of the said signature 1, 2 and/or family profile. For further guidance the skilled person will consider the known sequences of SGLT5, in particular hSGLT5.

The invention preferably relates to nucleic acids coding for at least one mammalian, more particularly human member of the SGLT co-transporter family.

The nucleic acid encodes at least one member of the SGLT co-transporter family having the function of sodium dependent co-transport of mannose and/or fructose. The skilled person can test for this function as outlined in more detail in this specification. Briefly, it is known that sodium ions are transported by the co-transporters along a concentration gradient, e.g. from the outside of a cell to the inside of a cell. The co-transport or symport of the solute occurs in the same direction and is functionally coupled to the transport of sodium. Thereby the solute can be transported against a concentration gradient, and/or across membranes which are otherwise impenetrable for the solute, e.g. plasma membranes. To evaluate the co-transport function, the skilled person can assay sodium flow and/or solute flow, as outlined in more detail herein.

In a preferred embodiment of the invention, the member of the SGLT co-transporter family encoded by the nucleic acid is SGLT5. The invention is not limited to, but preferably relates to mammalian, and more particularly human SGLT5. Known examples of SGLT5 include rabbit (U08813), human (AK057946), rat (XM_{—}220540, see also NM_{—}001107007.1) and bovine (AY514442) SGLT5 (Pajor, 1994, Leicht et al., 2006, Zhao et al., 2005a).

To the extent different splice variants exist for a sequence encoding SGLT5 according to any aspect of the invention, the invention preferably relates to splice variants encoding for a functional sodium dependent co-transporter for mannose and/or fructose. In particular embodiments, short splice variants that encode a non-functional protein are excluded.

In preferred embodiments the nucleic acid encoding SGLT5 encodes human SGLT5, and more preferably comprises or consists of a sequence according to SEQ ID No. 1, or comprises or consists of a sequence encoding the amino acid sequence according to SEQ ID No. 2. In a further embodiment the nucleic acid may comprise or consist of a sequence according to SEQ ID No. 3, i.e. the full mRNA sequence according to NM_{—}001042450.

The invention, however, is not limited to any of these specific examples of sequences.

The invention also relates to homologues of SGLT5, or variants related to SGLT5. The homologues or variants are characterized by having a nucleotide or amino acid identity of at least 65%, e.g. at least 70%, 75%, 80%, 85%, 90%, 95%, 98% or 99% with any of the specific sequences mentioned above, e.g. human SGLT5, more in particular with a sequence encoding the amino acid sequence of SEQ ID No. 2, even more in particular the nucleotide sequence of SEQ ID No. 1. The level of identity can be determined by routine means, such as the ClustalW or Blast software using standard settings. The invention also relates to sequences encoding homologues or variants of SGLT5, characterized by hybridizing under stringent conditions to the complement of any one of the nucleic acid sequences coding for a protein as described herein, e.g. human SGLT5, e.g. a protein according to SEQ ID No. 2, e.g. a protein encoded by SEQ ID No. 1. The skilled person is aware of suitable conditions for stringent hybridization. For example, the skilled person can chose the conditions such that sequences having a nucleotide homology of less than 65%, e.g. less than 70%, 80%, 90% or 95% are eliminated during the washing step. In embodiments of the invention the variants of specific nucleic acids as described herein are limited to nucleic acids encoding for a protein having sodium dependent co-transporter function for mannose and/or fructose, preferably a member of the SGLT family having this function.

There is only a relatively low level of sequence identity between SGLT5 and other members of the SGLT family. For example, Zhao et al., 2005b report that bovine SGLT2 and SGLT5 are 58% identical on the amino acid level. Human SGLT5 (NP_{—}001035915) shows 54% identity with human SGLT1. There is also a considerable level of intra-species variability. Zhao et al., 2005a report that bovine SGLT5 is 85, 64 and 48 identical to rabbit, human and rat SGLT5, respectively.

As used herein, the term “nucleic acid” can be understood in its broadest sense. It encompasses as non-limiting examples DNA, cDNA, RNA (both single and double stranded), and chemically modified variants thereof. Generally, nucleic acids of the present invention are compatible with host cells as defined herein. In particular, nucleic acids as understood in the context of this description can carry genetic information, e.g. in the form of a sequence coding for a protein or regulatory element. Thus, suitable chemical modifications are limited to such that do not compromise this sequence information. The skilled person is aware of nucleic acids and chemical derivatives thereof that can be used for introducing genetic information into host cells as defined herein.

The nucleic acid in particular can be in the form of a construct, plasmid or vector that can be used for introducing genetic information into a host cell. Specific examples of nucleic acids comprise vectors, plasmids, YACs or BACs. “Vector” is understood to be a general term that encompasses different nucleic acid formats, such as plasmids or recombinant viruses that can carry a nucleotide sequence, e.g. a DNA sequence, into a host cell and can there direct the synthesis of at least one specific protein. The skilled person is well aware of and can select from a wide range of known nucleic acid formats which are suitable for introducing genetic information into a host cell. For example, for recombinant expression in insect cells baculovirus can be used as a vector. In mammalian cells, non-viral or virus-based vectors can be used. Non-limiting examples of viral vectors include DNA viruses or retroviruses, e.g. adenovirus, adeno-associated virus, baculovirus, coronavirus, Epstein-Barr virus, herpes simplex virus, lentiviruses, poliovirus, retroviruses, semliki forest virus, simian virus 40, sindbis virus, vaccinia virus and others. The skilled person can select integrating vectors or such that remain extrachromosomally. The skilled person can also select vectors that integrate in a site specific manner or randomly into the host genome. The skilled person can also select vectors that exist as stable extrachromosomal elements, such as plasmids, yeast artificial chromosome (YAC) or bacterial artificial chromosome (BAC).

In some embodiments the skilled person will select vectors that are suitable for transient expression of the proteins of the present invention.

The nucleic acid of the invention comprises one or more nucleotide sequences coding for a protein having sodium-dependent cotransporter function for mannose and/or fructose. However, further nucleotide sequences may be present. It is conceivable that the sequence for the said protein comprising coding and non-coding sequences, e.g. exons and introns and/or untranslated 3′ or 5′ sequences. Moreover, the nucleic acids of the invention will in some embodiments comprise further nucleotide sequences, such as regulatory elements. Regulatory elements have an effect on the transcription of the sequence for a protein of interest. Typical non limiting examples are promoters and/or enhancers. The regulatory elements, such as promoters, are oftentimes located upstream of the coding sequence. The invention in particular relates to regulatory elements that allow for the inducible expression of proteins, e.g. by inducers as defined herein.

Examples of further nucleotide sequences comprise such that are associated with the amplification of the nucleic acid, its manipulation and introduction/function within a host cell. For example, nucleic acids such as plasmids or vectors oftentimes comprise at least one origin of replication that is suitable for one or more host cells, such that the nucleic acid can be amplified by a host cell. The nucleic acid of the invention may comprise one or more restriction sites which facilitate manipulations such as cleavage and ligation. The nucleic acids of the invention may comprise sequences that are related to introduction and/or function within a host cell. For example, the nucleic acids may comprise one or more sequences that relate to incorporation into the host genome, stabilize the plasmid or vector as an extrachromosomal nucleic acid, and/or influence copy number of the nucleic acid within the host cell. The nucleic acid of the invention may also comprise one or more nucleotide sequences facilitating selection of host cells that comprise the nucleic acid. Such sequences may be one or more selection marker, such as sequences coding for antibiotical resistance, sequences coding for optically detectable selection markers, such as fluorescent proteins, e.g. GFP, and/or sequences coding for a function that is deficient in the host cell, and is complemented by the introduced nucleic acid. Functions that are deficient in a host cell can relate to various aspects of metabolism which e.g. lead to auxotrophic cell strains. Introducing a nucleic acid into the host strain which complements the deficiency alleviates the auxotrophic feature and allows growth of the host cells under conditions wherein the factor for which the cell is auxotrophic is not present.

The nucleic acids of the invention in preferred embodiments comprise at lest the one or more sequences coding for a protein of interest, in particular a member of the SGLT co-transporter family. The nucleic acid sequence in advantageous embodiments is suitable for inducible expression. “Inducible expression” means that the protein is not constitutively expressed. There are many different forms of inducible expression the skilled person can choose from. Expression can be induced e.g. by changes in culture conditions such as e.g. culture temperature, and/or by addition and/or withdrawal of chemical compounds of the culture medium. Inducible expression systems include systems which can be switched on or off by one or more of the said changes. A wide range of inducible expression systems are known to the skilled person for the various host cells described herein. Widely used inducible expression systems are related to induction with tetracycline and other molecules, as explained above. More specifically, such examples include but are not limited to an inducer selected from a) Tetracyline or tetracycline derivative doxycycline (Tet-on or Tet-off), b) Ecdyson or muristerone A, c) Mifepristone, d) temperature shift acting on a temperature sensitive promoter, e) transient expression by adenovirus mediated gene transfer, or f) inducible expression by retrovirus mediated gene transfer, each e.g. as further described herein.

Thus, the skilled person knows a variety of commercially available inducible expression systems, such as several expression systems based on tetracycline induction, e.g. the T-Rex® system available from Clontech Laboratories (Mountain View, Calif., USA). Thus, in a particular embodiment the pT-Rex-DEST30 expression vector can be used to generate host cells expressing a protein of the present invention in a stable and inducible manner.

Thus, the nucleic acids of the present invention are suitable for inducible expression in a host cell. The introduction of a nucleic acid for expression of a protein is also referred to as “recombinant expression”. A nucleic acid is recombinant if it is formed by at least two nucleic acid sequences, e.g. DNA sequences, from different sources. For example, the combination of a plasmid or viral sequence with a sequence coding for a protein of interest represent classical examples of recombinant nucleic acids. As detailed above, the nucleic acid will harbour suitable regulatory elements to allow for inducible expression of the protein in a host cell.

Recombinant expression as used herein means that the protein encoded by the nucleic acid of the invention is formed by the host cell. Preferably the protein is correctly folded and integrated into the host cell membrane. Recombinant expression may also encompass posttranslational modifications, such as glycosylation.

Host Cells

The present invention relates to a wide range of host cells. Host cells suitable for recombinant expression include prokaryotic and eukaryotic hosts, such as bacteria, yeast, insect cells or mammalian cells all of them basically known to a person skilled in the art.

As the proteins of interest for the present invention are glycosylated, the host cells preferably are capable of glycosylating a recombinant protein. Such cells include yeast cells (which may optionally be genetically modified to adapt the glycosylation pattern, e.g. to a human glycosylation pattern), insect cells (such as lepidopteran cells and drosophila cells), vertebrate cells (such as Xenopus oocyces), more specifically mammalian cells. In preferred embodiments of the invention the host cells are mammalian cells, such as African Green monkey cells, such as VERO cells or COS cells, e.g. COS-1 or COS-7 cells, canine kidney cells, such as MDCK cells, or HEK cells, e.g. HEK293 cells, HeLa or CHO cells. In a particular embodiment of the invention the mammalian host cells are HEK cells, in particular HEK293 cells, commercially available e.g. by the Deutsche Sammlung von Mikroorganismen and Zellkulturen (DSMZ; Braunschweig, Germany) or the American Type Culture Collection (ATCC; Manassas, Va., USA). T-Rex Hek293 cells are commercially available from Invitrogen (see above).

The host cells of the invention comprise at least one nucleic acid as described herein. The skilled person knows suitable techniques for introducing the nucleic acids into the host cells, such as transfection and transduction techniques.

The host cells of the invention express, or are capable of expressing the member of SGLT co-transporter family having the function of sodium dependent co-transport of mannose and/or fructose in a stable and inducible manner.

In the context of the present invention stable expression means that the functional properties of the host cell, in particular the inducible functional properties associated with the member of SGLT co-transporter family having the function of sodium dependent co-transport of mannose and/or fructose, do not change over time, or do not change in a significant manner, if induced appropriately. This means that the host cells do not loose the co-transporter or fail to show co-transporter function after a certain time in culture (always under the presumption of optimal inducing conditions, e.g. optimal presence of an inducing chemical). Of particular interest in the context of the invention is that the cells maintain a stable inducible phenotype for multiple generations in culture. Thus, in particular embodiments, the functional properties of the cells do not change (or do not change significantly) over several cell cycles, e.g. 5, 10, 15 or 20 cell cycles. Cell cycles can be counted as of the time of introducing the nucleic acid of the present invention into the host cell, or any suitable time point thereafter, e.g. after thawing an aliquot of cells. As the inducible functional properties of the host cells of the invention remain stable, the precise number of cell divisions is not critical. For example, freshly thawed cells and cells that were kept in culture for a certain period of time will have the same or essentially the same functional properties. Cell cycles can e.g. be estimated on the basis of replication times of cells.

Each assay for inducible host cell phenotype, e.g. sodium dependent co-transport of mannose and/or fructose, shows a certain variability, e.g. intra-assay variability between certain samples or wells. The assays will typically also show a certain inter-assay variability, that is if the same assay is run with aliquots of the same batch of cells (e.g. several aliquots of thawed cells) this will result in a certain variability in readout. These kinds of variability are well known and are not associated with a loss of the protein in the cells and/or loss of function of the protein. Thus, the term “the functional properties do not change or do not change significantly” does not intend to exclude intra- or inter-assay variability that is commonly observed in cell based assays, and which is not associated with the loss of protein expression and/or loss of protein function.

“The functional properties do not change or do not change significantly” means that in a particular assay and after appropriate and comparable induction of expression, the readout of cells e.g. after different numbers of cell cycles (preferably in a direct comparison) does not differ by more than e.g. 20%, 15%, 10% or 5%. These values optionally are understood to describe a variation in addition to underlying assay variability (intra- and/or inter-assay variability).

Preferably the readout is identical, i.e. the comparison of cells after different numbers of cell cycles show values that are within the variability of the assay which is determined for cells having the same number of cell cycles.

The constitutive recombinant expression of SGLT5 in HEK cells has previously been reported (Leicht et al., 2006). However, the constitutive expression of SGLT5 rendered these cells unstable. This means that the functions associated with SGLT5 were lost or changed significantly when the cell line was maintained in culture. Thus, assay readouts obtained with these cells are not reproducible. Absent a consistent functional expression of SGLT5 in these cells, they were also unsuitable for establishing the proper biological function of SGLT5. As a matter of fact, Leicht et al. 2006 reports a function of SGLT5 which is not related to the actual function of this transporter as elucidated by the present inventors. Hence, stable inducible expression of SGLT5 function is a prerequisite for elucidating its function, and for any further aspects of the present invention, such as the methods, assays and kits of the present invention.

The physiological role of SGLT-5 in monosaccharide and sodium reabsorption and/or potentially pathological effects of abnormal, e.g. dysfunctional or absent expression of

SGLT5 in the uptake of monosaccharides in the kidney and into several tissues is not fully understood. Apart from SGLT-5, other members of the SGLT family are also known to be expressed in the kidney, and include e.g. SGLT-4, 5, and 6 (Tazawa et al., 2005).

The host cells of the present invention express, or are capable of expressing the member of SGLT co-transporter family having the function of sodium dependent co-transport of mannose and/or fructose in a stable and inducible manner. “In an inducible manner” means that the cells do not express the protein constitutively (see also above). It is assumed that constitutive expression leads to a selection against cells expressing the protein, or cells expressing the protein in a functional manner, or expressing the protein at levels that are functionally relevant. This negative selective pressure against cells constitutively expressing the member of SGLT co-transporter family having the function of sodium dependent co-transport of mannose and/or fructose, e.g. SGLT5, have hitherto made the elucidation of the function of this protein impossible, and have resulted in misleading and false positive results.

One specific example of a host cell of the present invention includes transfected T-Rex 293 cells inducibly expressing human SGLT5 which have been deposited under the Budapest Treaty with the DSMZ, Inhoffenstr. 7B, D-38124 Braunschweig, Germany, under the accession number DSM ACC3096. The depositor is Boehringer Ingelheim Pharma GmbH & Co KG, Birkendorferstr. 65, 88397 Biberach an der Riss, Germany.

Protein of the Invention and Nucleic Acids Encoding the Same

The present invention relates to proteins encoded by any of the nucleic acids described herein. These proteins are sometimes also referred to as “proteins of the present invention”. The proteins of the present invention have the function of sodium dependent co-transport of mannose and/or fructose, and in particular embodiments are members of the SGLT family of proteins. As the function of different members of the SGLT family are quite diverse, no prediction can be made on the function of any given family member.

Co-transport means that the transport of mannose and/or fructose is functionally coupled to transport of sodium ions at a fixed molar ratio. Cells typically maintain a lower sodium concentration than the extracellular space, e.g. by active transport of sodium from the inside of the cell to the outside. In sodium dependent co-transport by the proteins of the invention, sodium transport is typically along a concentration gradient, e.g. from the outside of a cell to the inside. The transport of mannose and/or fructose follows the same direction, e.g. from the outside of a cell to the inside, or from one side of a membrane to the other. The transport of mannose and/or fructose is thus not directly dependent on a concentration gradient of these sugars, nor on direct energy input (e.g. ATPase coupled transport). The transport also allows penetration of membranes which would not usually be penetrated by these sugars. Indirectly the energy for the transport is derived from the sodium gradient, maintained by input of energy.

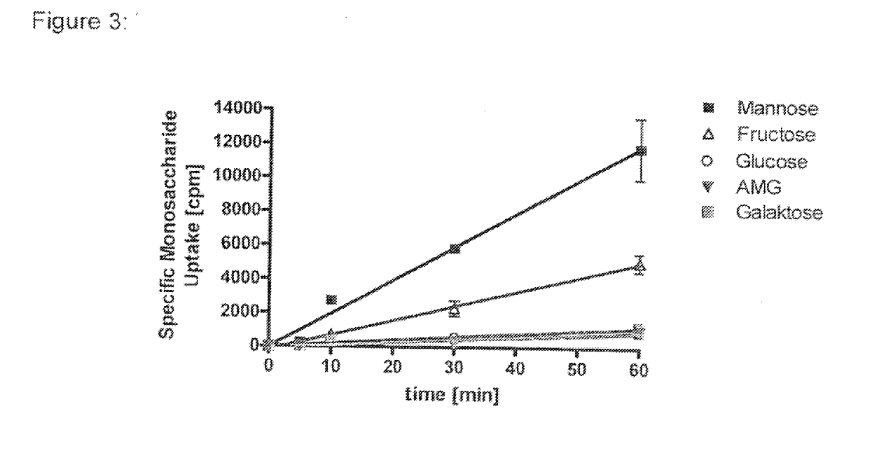

The proteins of the present invention have the function of sodium dependent co-transport of mannose and/or fructose. Initial work had suggested a function of galactose co-transport (Leicht S., 2005; Leicht et al, 2006). The art has also generally referred to SGLT5 as a glucose transporter (Gilbert et al., 2007), mostly, however, the art ref errs to SGLT5 as functionally uncharacterized. All the initial suggestions have now turned out to be wrong. As can be ascertained from the experimental section, SGLT5 does not exhibit notable galactose or glucose co-transport (see FIG. 3). The original reports possibly represented false positive results due to the instability and negative selection of the constitutively SGLT5 expressing HEK cells. The observed galactose transport likely was unspecific and remains irreproducible.

The proteins of the present invention specifically co-transport mannose and/or fructose. This means that the affinity of the co-transporter for mannose and fructose by far exceeds the affinity for other monosaccharides such as glucose, the sugar analogue alpha-methyl glucopyranoside (AMG) or galactose (see FIG. 3). Affinity can be directly measured by pharmacological techniques. For example, the affinity for mannose may be at least 5 fold, 10 fold or 15 fold higher for mannose as compared to glucose, AMG or galactose. At the same time, affinity for mannose is higher than affinity for fructose, e.g. 1 fold, 1.5 fold, 2 fold 2.5 fold or 3 fold higher.

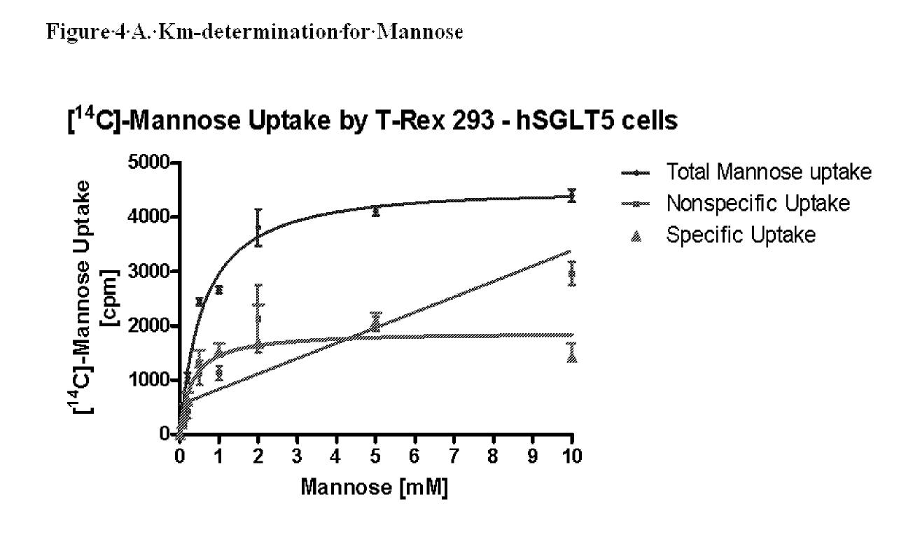

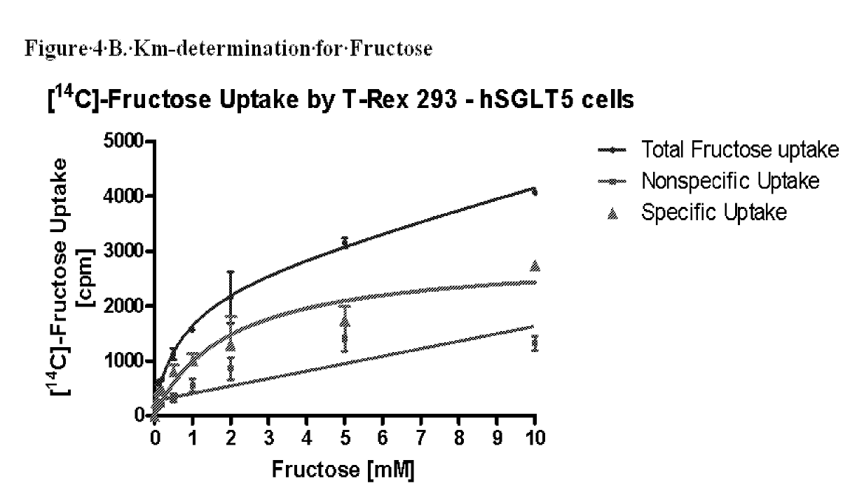

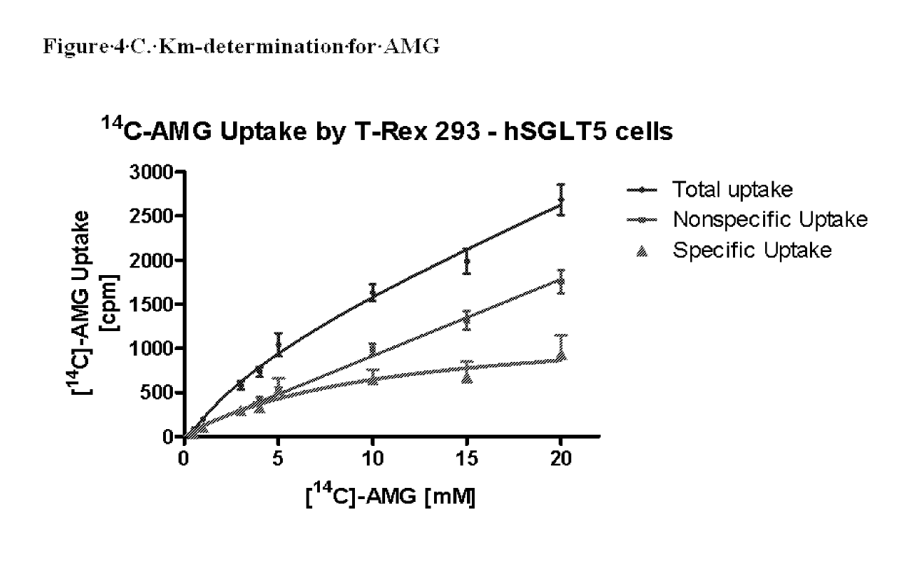

More specifically and as exemplified in the experimental section of this description (see also FIG. 4), the proteins of the present invention show selectivity for mannose in terms of the Km of mannose co-transport. The Km for mannose co-transport of proteins of the invention is below 1 mM, e.g. below 0.5 mM, more specifically 0.3 mM or less.

Amongst all sugars tested, the proteins of the invention showed the highest affinity/lowest Km for mannose.

The proteins of the invention also co-transport fructose with a low Km, which, however, is significantly higher than the Km for mannose (i.e. the co-transporter has a lower affinity for fructose as compared to mannose). The Km for fructose co-transport of proteins of the invention is below 5 mM, e.g. below 3 mM, more specifically 2.0 mM or less. Thus, the Km for fructose is 5 to 10 fold higher than the Km for mannose, more specifically 6 fold higher, e.g. 6.67 fold higher.

The proteins of the invention also co-transport AMG, however, with a significantly higher Km as compared to either mannose or fructose. More specifically, the Km for AMG is at least 15 mM, e.g. at least 12 mM, more specifically at least 10.1 mM. Thus, the Km for AMG is at least 25 fold higher than the Km for mannose, e.g. at least 30 fold higher, more specifically more at least 33 fold higher. The Km for AMG is at least 3 fold higher than the Km for fructose, e.g. at least 4 fold higher, more specifically at least 5 fold higher.

These data illustrate that SGLT5 unexpectedly acts as a specific co-transporter for mannose and/or fructose, with a clear preference for mannose over fructose. Other sugars, such as AMG, are transported with 20-30 fold lower affinities than mannose. This specificity for mannose (and/or fructose) was unexpected in view of the reports in the literature of a putative galactose transport by SGLT5. Other sugars such as galactose or glucose will have similar affinities to the protein of the invention as AMG, or even lower affinities (i.e. higher Km).

In terms of function affinity correlates with co-transport. This means that the co-transporter shows by far greater transport of mannose and fructose as compared with other monosaccharides such as glucose, AMG or galactose over the same period of time (see e.g. FIG. 3 and FIG. 4). For example, the difference in transport may be at least 5 fold, 10 fold, 15 fold or 20 fold when comparing mannose with any one of glucose, AMG or galactose. At the same time, the transport of mannose exceeds that of fructose. For example the difference in transport may be at least 1 fold, 1.5 fold, 2 fold, 2.5 fold or 3 fold when comparing mannose and fructose.

The present invention for the first time provides a function for the proteins of the present invention, in particular SGLT5, as a sodium dependent co-transporter of mannose and/or fructose. Based on this function, the skilled person can devise a wide variety of uses, methods and assays as well as related kits, assay systems and articles of manufacture as described herein. The function of the proteins of the invention can be tested by any of the methods and assays as described in the following.

Methods/Assays

In the context of the present invention methods and assays are used interchangeably.

The invention provides methods of assessing the transport of a compound, comprising the use of a nucleic acid, protein and/or host cell according to any aspect of the invention, or a cell obtainable by any aspect of the invention. The term “compound” is to be understood broadly, and includes collections or libraries of compounds. Examples of classes of substances falling under the term “compound”, as well as specific exemplary compounds are provided in the following. Non limiting examples of compound classes are macromolecules, such as polypeptides, e.g. antibodies or antibody fragments, but also nucleic acids or nucleic acid derivatives suitable for interaction with proteins of the invention. Further classes are compounds that are, or are related to the molecules co-transported by the protein of the invention, i.e. sodium, mannose and/or fructose. Such classes are metal ions, e.g. earth alkali metal ions, or sugars as defined above, including imino sugars, in particular monosaccharides or disaccharides, such as monosaccharides that are structurally related to mannose and/or fructose. Further classes of compounds include small molecular weight entities as defined above, in particular small molecular weight entities known to interact with SGLT family members (in particular SGLT5).

A large number of compounds interacting with different SGLT family members is known, and include the solutes co-transported by these proteins, e.g. mannose and/or fructose. It is conceivable that structural derivatives of such compounds can provide collections or libraries of compounds that can be tested for interaction with the proteins of the invention. Specific examples include phlorizin or Remoglifozin, which are known to interact with SGLT5 and inhibit its function. Phlorizin is the first prototypic SGLT inhibitor, a natural non-selective SGLT inhibitor. Either of these inhibitors can be used in proof of concept studies in the assessment of function of the proteins of the present invention.

Structural derivatives/analoges of any of the above compounds are also suitable candidates for libraries or collections of compounds that can be used in testing for interaction with the proteins of the present invention. Any examples of compounds or compounds classes named in this description is understood to complement these examples. Other lists of compounds are not meant to be limiting, but are understood as exemplary and can be read in combination with any other list provided herein, e.g. the above examples of compound classes or compounds.

The invention also provides a method of screening for a compound capable of interacting with a protein encoded by a nucleic acid according to any aspect of the invention. “Screening” as used herein means that at least one compound, but preferably a multitude of compounds, e.g. a collection or library of compounds of unknown function are tested for interaction, e.g. binding and/or function with the protein of the invention. For assessing binding to the protein of the invention, classes of compounds that are known for their ability to interact with proteins can be utilized. An important class includes antibodies and antibody fragments which show antigen binding. In this connection, the protein of the invention represents an “antigen”. Preferably the protein of the invention is in its native conformation, e.g. as a functional protein expressed in a host cell. Cell based methods of screening for antibodies or antibody fragments binding to an antigen are known in the art. However, the invention also considers the use of fragments or peptides derived from the protein of the invention as antigens. Furthermore, binding to the protein of the invention can be assessed by known pharmacological techniques, e.g. using labelled compounds, such as labelled (e.g. radioactively labelled) sugars as defined herein.

The invention also provides methods for assessing the function of a compound interacting with a protein encoded by a nucleic acid according to any aspect of the invention. “Assessing the function of a compound” means that one or more functions of the protein of the invention are tested, e.g. one or more of sodium transport, mannose transport and/or fructose transport.

Any of the methods of the invention may comprise the use of a host cell according to any of the aspects of the invention or obtainable by any method of the invention. To test interaction and/or functional effects on the protein of the invention, the host cells must be suitably induced to express the protein. The methods then comprise a step of a) contacting a cell expressing a protein encoded by a nucleic acid according to any aspect of the invention with one or more compounds and b) assessing one or more of binding to the protein and transport of a compound. The compound which is contacted with the protein of the invention and the compound the transport of which is tested may be the same or different. For example, if only binding is assessed, one assay format will include a selection of test compounds, e.g. a collection or library of compounds of which binding to the protein is unknown. In a further assay format, which can optionally be used in combination with the first assay format, the selection of test compounds is used in combination with one or more of the compounds known to interact with the protein of the invention, i.e. one or more of sodium, mannose and/or fructose. In this case binding and the potential competition with any of the compounds known to interact with the protein can be tested.

Thus, the invention encompasses the use of more than one compound at the same time. For example, a sugar which is co-transported, e.g. mannose and/or fructose, is tested in the presence of one or more further compounds as described herein in a competitive format. The changes in co-transport of e.g. mannose and/or fructose caused by said one or more further compounds can indicate the function of the one or more further compounds and/or the type of interaction with the protein of the invention. For example, if the compound is a further sugar as defined herein, a decrease in the co-transport of e.g. mannose may indicate that the further sugar is either competing for co-transport by the protein, or is inhibiting co-transport. These different kinds of interaction with the protein of the invention can readily be distinguished by routine pharmacological tests, e.g. by using a label in the one or more further compounds that are tested, and assaying the transport of the labelled compound by the protein.

Thus, in particular embodiments, the methods of the invention are based on a functional readout, i.e. a co-transport function is tested. Co-transport allows for a broad spectrum of assays and readout systems to test the function of the proteins of the invention.

Sodium influx results in an inward current into cells, and hence can be detected by electrophysiological means, e.g. the monitoring of a membrane potential or membrane currents. The skilled person knows suitable electrophysiological assays, including intracellular recordings, voltage clamp recordings, or patch clamp techniques. An exemplary, non limiting description of electrophysiological methods can be found in Bissonnette et al., 1999, who describes electrophysiological recordings on SGLT1 expressing cells. As host cells Xenopus oocytes were used. However, the assay can also be readily adapted to suitable host cells as described herein, e.g. mammalian host cells. The solute investigated in the assay of Bissonnette et al., is glucose, the solute co-transported by SGLT1. This can be changed to any of the substrates of the protein of the present invention, most notably mannose and/or fructose. To the extent other compounds (e.g. other sugars or small molecular weight entities) are tested, the assay will typically also include mannose and/or fructose as a positive control. Moreover, the inhibitor remoglifozin or the non-specific inhibitor phlorizin can be included as a further control in the assay. Remoglifozin has an IC50 of 190 nM on human SGLT5, phlorizin has an IC50 of 1500 nM (see also table 2). The skilled person knows meaningful readout values fur such assays. For example Km for a substrate and Ki for an inhibitor can be determined.

In one particular embodiment, the changes in membrane potential associated with the sodium currents can be assessed. Apart from electrophysiological recordings, the skilled person also knows of indirect methods for assessing membrane potential. For example, the skilled person knows fluorescent dyes that respond to changes in membrane potential by changing their fluorescent emission. There are commercial assays, such as the FLIPR tetra assay of Molecular Devices that can be used by the skilled person. Specific examples of suitable experimental paradigms are described in S. Leicht, 2005, or Leicht et al., 2006.

Alternatively or in addition the skilled person can measure the influx of mannose and/or fructose. The skilled person knows suitable assays for this purpose. For example, labelled monosaccharides can be employed. The type of label is not limited, provided it does not interfere with transport function. For example, radioactively labelled monosaccharides can be employed, e.g. C14 labelled mannose and or fructose. Suitable saccharide analogues or small molecule entities can also be employed.

The invention includes embodiments in which the sugar, sugar analogue or small molecular weight compound is transported by the co-transporter. In this case, the methods can either determine transport of these compounds alone, or represent competitive assays wherein the compound competes with certain amounts of mannose and/or fructose.

The invention also includes embodiments in which the sugar, sugar analogue or small molecular weight compound positively or negatively affect co-transport, i.e. enhance or inhibit co-transport. In this instance the compounds act as any one of agonists, partial agonists, inverse agonists, or antagonists of the proteins of the invention. For example, a co-transport inhibitor can be used as a control, and/or competitor in the assays to measure co-transport. The outline of a suitable assay is explained e.g. in Tazawa et al., 2005, Leicht et al., 2006 or exemplified in the experimental section of this description. Componds known to inhibit co-transport by the proteins of the invention include phlorizin (IC50 1500 nM), or remoglifozin (IC50 190 nM). Remoglifozin has been described as a SGLT2 inhibitor by Fujimori et al., 2008. Remogliflozin etabonate is a prodrug based on benzylpyrazole glucoside and is metabolized to its active form, remogliflozin, in the body.

In one exemplary embodiment, a method of measuring co-transport utilizing labelled solute can be described as follows. Cells harbouring a nucleic acid encoding a protein of the invention are induced, exposed to a monosaccharide or other compound which is labelled, and incubated. Thereafter the cells can be analyzed for uptake, e.g. by washing and lysing the cells and quantification of transported label (e.g. counting radioactivity).

For example, a suitable number of cells harbouring a nucleic acid of the invention, i.e. a nucleic acid suitable for stable, inducible expression of a protein of the invention e.g. 10-50.000 cells, such as 30.000 cells can be seeded per well in 96 well plates, which preferably are coated, e.g. with poly-D-lysine. The expression of the protein can be induced at a suitable time before the start of the co-transport experiment, such as 36, 32, 24, 18 or 16 hours before the start. Induction depends on the inducible expression system used, and can be effected e.g. by addition of a suitable amount of tetracycline in a tetracycline inducible system, e.g. 0.5-1.5 μg/ml, such as 1 μg/ml, to the cells. For the assay the cells preferably are washed in a suitable buffer, e.g. in a suitable volume such as 250 μl of pre-treatment buffer. A particular example of a suitable pre-treatment buffer is provided in the experimental section; such pre-treatment is recommended for a preferred mode of the invention. The cells are then incubated in an appropriate volume of pre-treatment buffer, e.g. 150-250 μl, such as 200 μl for a specified period of time, such as 20, 25 or 30 min e.g. at 37° C. After removal of the pre-treatment buffer, the uptake buffer is added to the cells (for a specific example of a suitable uptake buffer see experimental section). The uptake buffer contains an unlabelled monosaccharide, e.g. unlabelled mannose, and in addition contains a certain amount of the same monosaccharide that is labelled, e.g. radioactively labelled. For example, the uptake buffer may comprise a suitable amount, e.g. 0.5-5 mM, such as 1 mM of any on of glucose, fructose, mannose, galactose or AMG, or any other saccharide or small molecular weight entity and furthermore contain a suitable amount of the respective labelled (e.g. C14 labelled) monosaccharide, e.g. between 0.2 and 1 μCi; e.g. 0.6 μCi. The assay may involve auxiliary substances that have a beneficial influence on specificity and sensitivity. For example, together with the monosaccharides Cytochalasin B can be added, e.g. 5-15 μM, such as 10 μM. The cells can then be incubated for an appropriate amount of time (e.g. between 5 and 90 min, such as 5, 10, 30, 60 or 90 min, or even up to 3, 6, 12, 18 or 24 h), washed (e.g. with PBS) and optionally lysed to determine the uptake into the cells.

Thus, in certain aspects the methods of the present invention focus on the transport of compounds by the proteins of the present invention. The transport per se can be assayed as outlined herein, e.g. by measuring sodium flux and/or mannose and/or fructose import. However, the methods also extend to assaying further compounds that interact with the proteins as described herein.

“Interact” is understood in its broadest sense, and includes binding as well as binding combined with functional effects, e.g. agonistic or antagonistic effects.

The invention relates to assaying interaction of compounds with respect to sodium, i.e. compounds that compete with sodium, can take the place of sodium, are transported like sodium, or inhibit or stimulate transport of sodium. In this regard, the invention considers assaying compounds that resemble sodium, e.g. metal ions, more particular earth alkali metal ions, specifically such ions carrying a single positive charge. However, the invention also considers compounds that are no metal ions, including small molecular weight chemical entities, nucleic acids and polypeptides, such as antibodies or antibody fragments and peptidomimetics. Metal ions or other compounds may interact with the co-transporter directly at the site where sodium interacts with the protein, or indirectly at another site.

Alternatively, or in addition, the invention relates to assaying interaction of compounds with respect to mannose and/or fructose, i.e. compounds that compete with these monosaccharides, can take their place, are transported like them, or inhibit or stimulate their transport. The compounds can be other saccharides, in particular monosaccharides, or chemical analogues, such as AMG, 1,5-anhydro-D-glucitol (1,5AG), 2-deoxy-D-glucose (2DG), 3-0-methyl-D-glucose (30MG). They can also be small molecular weight chemical entities, nucleic acids and polypeptides, such as antibodies or antibody fragments and peptidomimetics. Any such compound may interact with the co-transporter directly at the site where mannose and/or fructose interacts with the protein, or indirectly at another site.

Thus, the invention relates to methods and assays for screening the interaction of compounds as described above with the proteins of the invention. In this embodiment, or any other embodiment, the present invention also relates to contacting the protein of the invention with collections or libraries of compounds in a suitable format, e.g. a high throughput format. Collections or libraries can be randomly assorted, or can be based on derivatives or analogues of the solutes co-transported by the proteins of the present invention, in particular of mannose and/or fructose, or derivative or analogues of compounds found to interact with and/or inhibit the protein of the present invention, e.g. plorizin or remogliflozin.

The methods of the present invention may be based on binding assays alone. For example, binding to a protein of the present invention can be determined by assays such as ELISA, RIA, FACS or cell based assays, e.g. phage panning, etc. Binding can also be determined e.g. by Biacore assays or by radioligand binding assays as demonstrated in the experimental part below. However, as the protein of the present invention is a complex transmembrane protein, membrane stemming protein is preferably used for any of the methods as described herein. In particular cell based protein can be used in the assays, methods or uses of the invention.

However, any of the above embodiments can also be performed as cell free methods, uses or assays wherein the transmembrane protein is inserted in a suitable membrane (e.g. derived from cells of the invention) or membrane analogue, i.e. a lipid (in particular phospholipid) layer, e.g. bilayer, that is not derived from cells but encompasses the protein of the invention (e.g. liposomes). Binding can be determined by cell based assays, or cell free assays, in particular high throughput cell based or cell free assays.

In other embodiments, the analysis of binding and the analysis of function can be coupled. For example, dyes responsive to changes in membrane potential can be employed in cell based assays of function as described herein, e.g. in high-throughput cell based assays for screening binding and function of collections or libraries of compounds.

Any embodiment of the present invention relates to the different classes of compounds or examples of compounds as provided herein, without limitation. Thus, any of the methods or assays can be read in combination with any compound or class of compound as specified in this description.

Kits, Assay Systems and Articles of Manufacture

The present invention also relates to kits, assay systems and articles of manufacture which comprise subject matter as defined herein, e.g. one or more of nucleic acids, proteins or host cells as described herein.

The present invention also relates to kits, assay systems and articles of manufacture which are suitable for and/or specifically adapted for and/or useful in any method or assay as described herein. The kits, assay systems or articles of manufacture may also comprise subject matter which is unrelated to the nucleic acids, proteins or host cells of the invention, either alone or in combination with any such subject matter of the invention, provided the kits or articles of manufacture are suitable for and/or specifically adapted for and/or useful for a method of the present invention. The kits or articles of manufacture may also comprise instructions for the user and/or may comprise packaging material. In certain embodiments the kits or articles of manufacture will comprise reagents necessary for performing any of the methods of the present invention. In certain embodiments the kits or articles of manufacture will comprise control reagents, e.g. a positive control and/or negative control that is suitable for testing sodium dependent co-transport of mannose and/or fructose. The kits or articles of manufacture may also comprise reagents and/or dilution series suitable for a standard curve. As positive control or standard curve mannose and/or fructose (e.g. at suitable predefined concentrations, e.g. suitable for dilution into a standard curve or as a prepared final concentration of a standard curve) can be used. As negative control e.g. inhibitors such as phlorizin or Remoglifozin can be used.

The present invention also relates to uses of the nucleic acids, proteins, host cells of the invention or cells obtainable by methods of the invention. The uses include uses in methods and assays, e.g. use in screening assays, assays for binding, assays for function, or any combination thereof. For example, the T-Rex 293-hSGLT5 cell line could be used to identify further substrates of this transporter, to reveal the physiological role of the transporter and also to identify inhibitors or activators of the transporter.

Experiments

In the following, specific exemplary aspects of the present invention are described by reference to examples. The invention is not understood as being limited to these examples. Rather the skilled person understands these examples in the context of, and specifically in combination with the general parts of the description.

### EXAMPLE 1

Cloning of SGLT5 from human kidney: Human SGLTS was cloned from kidney mRNA by reverse transcription and PCR. A transcript of 1980 kb representing a protein of 597 amino acids was cloned into the pcDNA3.1/V5-His-TOPO plasmid. The coding sequence was subsequently cloned into the pENTR/D-TOPO® plasmid (Invitrogen, Carlsbad, Calif., USA) (Forward primer: 5′-CAC CGC CAT GGC CGC CAA CTC CA-3′ (SEQ ID No. 4) and Reverse primer 5′-TCA GGC GAA GTA GGC ATA AAA GAA TAT GTT GAC-3′ (SEQ ID No. 5)) using the TOPO® TA cloning kit (Invitrogen, Carlsbad, Calif., USA) and ligated into the pT-Rex-Dest30 plasmid via the clonase reaction.

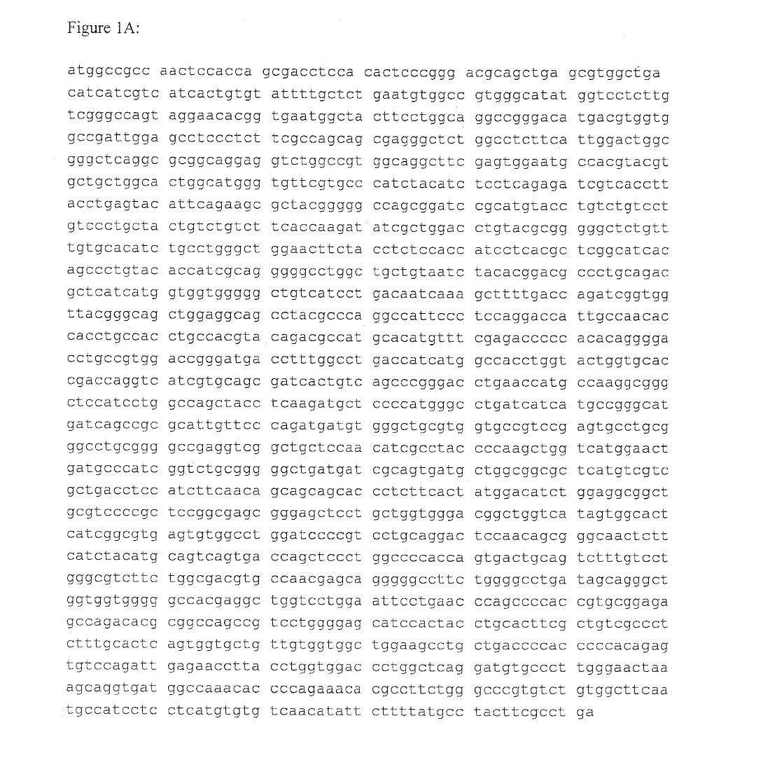

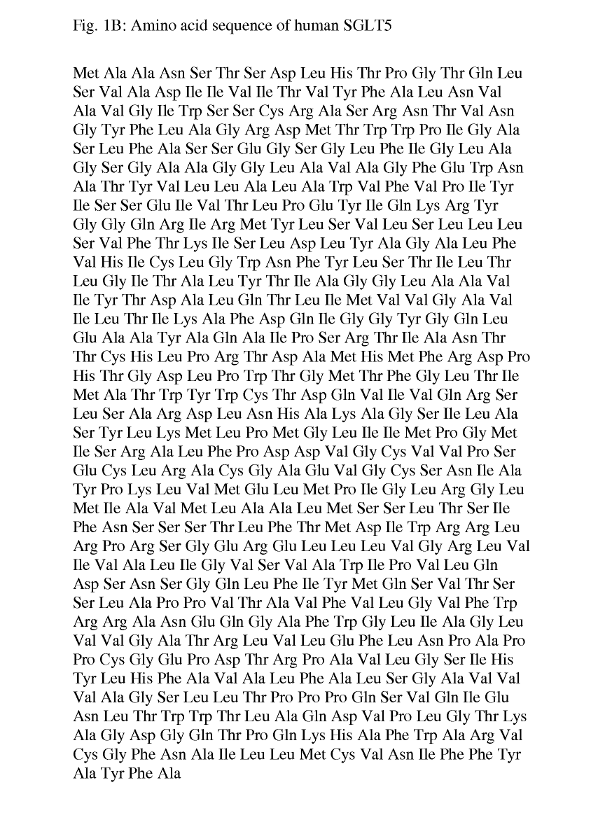

The insert was sequenced and the sequence was aligned to the published hSGLT5 sequence (Acc.No. NM_{—}001042450). The cloned hSGLT5 variant has an open reading frame of 1791 bp. The coding sequence extends from nucleotide 42 to 1832 and is shown in FIG. 1A (SEQ ID No. 1). The full mRNA sequence according to NM_{—}001042450 is shown as SEQ ID No. 3.

Generation of stable inducible T-Rex 293-hSGLT5 cells: The pT-Rex-Dest30-hSGLT5 plasmid was transfected into T-Rex 293 cells (Invitrogen, Carlsbad, Calif., US) and stable cell clones were selected by limited dilution methodology and functional testing of clones using [¹⁴C]-saccharide uptake experiments (as described herein). T-Rex 293-hSGLT5 cells were cultivated in DMEM containing 10% FCS, 600 μg/ml geneticin and 5 μg/ml blasticidin.

Real-Time PCR experiments: RNA was prepared from T-Rex 293 cells or T-Rex 293-hSGLT5 cells and reversibly transcribed into cDNA using the High Capacity cDNA Archive Kit (Applied Biosystems, Foster City, Calif., US). This cDNA was used for Real-time PCR using specific FAM-labeled primers for SGLT5 (Forward primer: 5′-GAT GAC CTT TGG CCT GAC CAT-3′ (SEQ ID No. 6) and Reverse primer 5′-GTC CCG GGC TGA CAG TGA T-3′ (SEQ ID No. 7)) and VIC-labeled primers for GAPDH (Forward primer: 5′-TGG CAT GGA CTG TGG TCA-3′ (SEQ ID No. 8) and Reverse primer 5′-GCA CCA CCA ACT GCT TAG C-3′ (SEQ ID No. 9)). The amount of mRNA of hSGLT5 was normalized by the expression of GAPDH for each sample.

FLIPR assay: Changes in membrane potential can be assayed using the FLIPR TETRA Assay (Molecular Devices). In this method cells are loaded with a fluorescent dye, which emits light of a different wavelength dependent on the membrane potential. This can be recorded in suitable cell culturing plates and monitored over time in the presence/absence of a test substance.

10.000 to 30.000 cells were seeded in a 384 well plate (black, lysine coated with transparent bottom, Becton Dickinson) and were incubated over night at 37° C. and 5% CO2. The next day they were washed twice with 50 μl assay buffer (140 mM NaCl; 1.26 mM CaCl₂; 0.49 mM MgCl₂*6 H₂O; 0.41 mM MgSO₄*7 H₂O; 5.33 mM KCl; 0.44 mM KH₂PO₄−pH =7.4). Consecutively the following was added: 20 ml assay buffer, 20 ml assay buffer with or without inhibitor, 20 ml fluorescent dye (membrane potential assay RED, Molecular Devices) dissolved in assay buffer. Thereafter the plates were incubated for 30 minutes at 37° C. and 5% CO2. Before beginning the measurement the settings of the FLIPR analyzer were set such that the average number of counts in the test was in the range of 2,500-4,000.

After starting the measurement ten sample points were assayed at intervals of 3 seconds. Thereafter 20 ml of the dissolved test substances (e.g. monosaccharide and/or KCl) were added and 30 measurements were recorded in 3-second intervals, thereafter 16 measurements were recorded in 15-second intervals.

[¹⁴C]-saccharide uptake experiments: 30000 cells per well were seeded in white poly-D-lysine-coated 96-well plates and incubated over night. The expression of hSGLT5 was induced 24 h before the start of uptake experiments by the addition of 1 μg/ml tetracycline to the cells. For the assay, cells were washed twice with 250 μl pre-treatment buffer [140 mM cholinchloride in basal buffer (10 mM HEPES; 5.4 mM KCl; 2.8 mM CaCl₂*2 H₂O; 1.2 mM MgSO₄*7 H₂O; 0.1% BSA, pH 7.4) and incubated in 200 μl pre-treatment buffer for 25 min at 37° C. Pre-treatment buffer was removed and 200 μl uptake buffer was added to the cells (140 mM NaCl in basal buffer containing 1 mM cold monosaccharide [either glucose, fructose, mannose, galactose or alphamethyl glucopyranoside (AMG), respectively]+0,6 μCi [¹⁴C]-labelled monosaccharide [either glucose, fructose, mannose, galactose or alpha-methyl glucopyranoside (AMG), respectively] (Hartmann Analytics, Mannheim, Germany) and 10 μM Cytochalasin B. After incubation for 5, 10, 30 and 60 minutes at 37° C., the cells were washed three times with 300 μl PBS (Cambrex, Verviers, Belgium) and then lysed in 0.1 N NaOH with intermittent shaking for 5 min. The lysate was mixed with 200 μl MikroScint 40 Scintillator, shaken for 15 min and counted for radioactivity in the TopCount NXT (Canberra Packard, Schwadorf, Austria). Specific sugar uptake was determined by subtracting non-specific uptake of each sugar (in the presence of choline chloride) from total uptake of the same sugar (in the presence of sodium chloride).

[¹⁴C]-monosaccharide uptake inhibition experiments: Stable cell lines overexpressing human SGLT-5 were used for the sodium-dependent monosaccharide transport inhibition assay. Cells were pre-incubated in 200 μl uptake buffer (10 mM HEPES, 137 mM NaCl, 5.4 mM KCl, 2.8 mM CaCl₂, 1.2 mM MgCl₂, 50 μg/ml Gentamycin, 0.1% BSA) for 25 minutes at 37° C. 10 μM Cytochalasin B and test compound was added at different concentrations 15 minutes before the initiation of the uptake experiment.

The uptake reaction was started by the addition of 0.6 μCi [¹⁴C]-labelled monosaccharide i.e. [¹⁴C]-labeled alpha-methyl glucopyranoside (AMG), glucose, fructose, mannose or myo-inositol, in 0.1 mM AMG (or the respective non-radioactive monosaccharide).

After incubation for 60 minutes at 37° C., the cells were washed three times with 300 μl PBS (Cambrex, Verviers, Belgium) and then lysed in 0.1 N NaOH with intermittent shaking for 5 minutes. The lysate was mixed with 200 μl MicroScint 40 and shaken for 15 minutes and counted for radioactivity in the TopCount NXT (Canberra Packard, Schwadorf, Austria). The cells were pre-incubated in pre-treatment buffer (uptake buffer containing choline chloride instead of sodium chloride) for 25 minutes prior to addition of uptake buffer.

A dose-response curve was fitted to an empirical four-parameter model using XL Fit (IDBS, Guildford, UK) to determine the inhibitor concentration at half-maximal response (IC₅₀).

Results:

Human SGLT-5 was identified to be a mannose transporter and the inhibition of hSGLT-5 was assessed by a newly established [¹⁴C]-mannose uptake assay.

The cDNA for a new human SGLT5 was cloned from human kidney mRNA. This cDNA was aligned to the mRNA described under ATCC accession number NM_{—}001042450 (Homo sapiens solute carrier family 5 (sodium/glucose cotransporter, member 10 (SLC5A10), transcript variant 2, mRNA) (SEQ ID No. 1; FIG. 1A). The corresponding amino acid sequence is shown in FIG. 1B (SEQ ID No. 2).

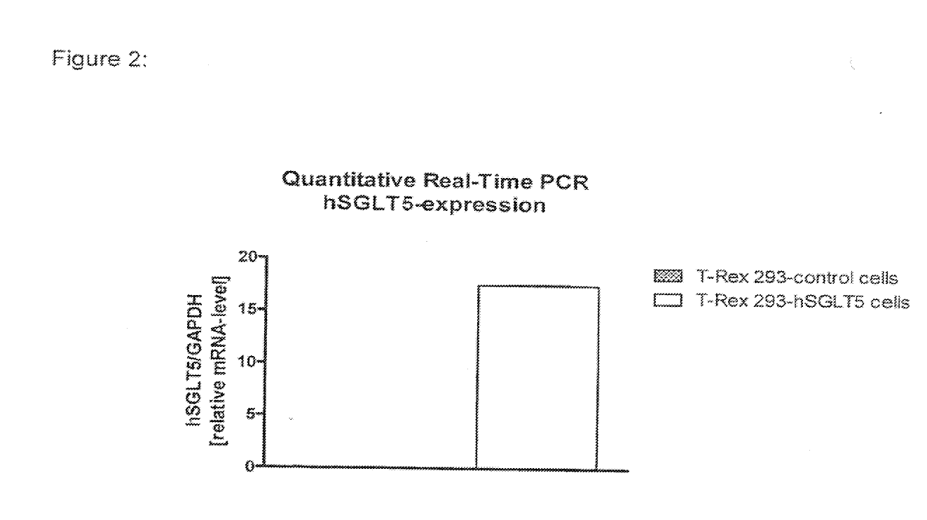

In further experiments the cDNA was subcloned into the expression vectors pT-Rex-DEST30 that was used to generate a stable cell line for inducible expression of human SGLT5 (T-Rex 293-hSGLT5). These cells showed a consistent and reproducible expression of human SGLT5 upon induction by tetracycline for 24 hours (FIG. 2). One specific clone of transfected T-Rex 293 cells inducibly expressing human SGLT5 has been deposited under the Budapest Treaty with the DSMZ, Inhoffenstr. 7B, D-38124 Braunschweig, under the accession number DSM ACC3096. The depositor is Boehringer Ingelheim Pharma GmbH&CoKG, Birkendorferstr. 65, 88397 Biberach an der Riss.

FIG. 2 shows the mRNA expression of human SGLT5 in T-Rex 293-hSGTL5 cells in comparison to non-transfected T-Rex 293-control cells relative to the house keeping gene glycerine aldehyde phosphate dehydrogenase (GAPDH) multiplied with 1×10⁶.

Furthermore, a protocol was established to measure the specific uptake of monosaccharides e.g. mannose, fructose, glucose, AMG and galactose by hSGLT5, It could be shown that the transporter takes up mannose and fructose with high affinity and hardly transports glucose, AMG and galactose (FIG. 3). FIG. 3 shows the time-dependent specific uptake of different monosaccharides by T-Rex 293-hSGLT5 cells. Specific uptake was calculated by subtracting the respective monosaccharide uptake in the presence of choline chloride from the total uptake of the same monosaccharide in the presence of sodium chloride. Mannose is the substrate with the highest affinity for hSGLT5. The transporter has also high affinity for fructose but very low affinity towards glucose, AMG or galactose.

Km-determination for mannose, fructose and Alpha-Methylglucose (AMG) uptake by SGLT5: The uptake of Mannose, Fructose and AMG was assayed as described under “[¹⁴C]-monosaccharide uptake inhibition experiments” in the absence (nonspecific uptake) or presence of sodium (Total uptake) in the buffer at different concentrations of the respective monosaccharide for 4 h. From the difference of Total uptake and Nonspecific uptake the Specific uptake was calculated and the Km was determined using the Program Graph Pad Prism (LaJolla, Calif., USA).

The measured affinity curves measured in three assays run in parallel are shown in FIG. 4A (for mannose), 4B (for fructose) and 4C (for AMG). The affinity values derived from these curves are summarized in table 1:

TABLE 1
Overview of the affinity for SGLT-5 of different sugars

| Sugar | Affinity for SGLT5 (Km [mM]) |
|---|---|
| Mannose | 0.3 |
| Fructose | 2.0 |
| AMG | 10.1 |

Native, untransformed Hek cells do not transport mannose because they do not express a respective transporter (data not shown). But these results show that these cells which were induced to express SGLT5 unexpectedly do transport mannose, which must be due to the SGLT5 activity. The affinity values as measured herein increase from AMG over fructose to mannose.

Thus, a novel assay for hSGLT-5 was established. SGLT-5 was overexpressed in T-Rex 293 cells and was shown to transport mannose, fructose and, to a lower extent, AMG.

In saccharide uptake inhibition experiments using a panel of C-glucosides and O-glucosides known to inhibit some members of the SGLT family, most inhibitors showed only a low inhibitory action on SGLT5. Only the O-glucoside remogliflozin exhibited a potent inhibition of SGLT-5 with an IC₅₀ of 190 nM and <15-fold selectivity over another member of the SGLT family, SGLT-2. An overview of the results is shown in the following table 2.

TABLE 2
Overview of potency on SGLT-5 of
structurally known SGLT inhibitors.

| | Mean IC₅₀ | (pIC₅₀ ± SEM) |
|---|---|---|
| C-glucosides | | |
| Compound A | 1100 | −5.98 ± 0.15 |
| Dapagliflozin | 820 | −6.09 ± 0.22 |
| Canagliflozin | 1700 | −5.77 ± 0.12 |
| ASP-1941 | 740 | −6.13 ± 0.11 |
| O-glucosides | | |
| Sergliflozin | 1100 | −5.95 (n = 1) |
| Remogliflozin | 190 | −6.72 ± 0.19 |
| T1095A | 1100 | −5.97 ± 0.19 |
| Phlorizin | 1500 | −5.82 ± 0.18 |

Results are shown as mean IC₅₀ [in nM] and pIC50 ± SEM for inhibition of hSGLT-5. [¹⁴C]-mannose was used as substrate. Data are derived from at least three independent experiments. Compound A is D-glucitol, 1,5-anhydro-1-C-[4-chloro-3-[[4-[[(3S)-tetrahydro-3-furanyl]oxy]phenyl]methyl]phenyl]-, (1S)-; ASP-1941 is a compound which has been published under this name by Astellas Pharma Inc. (JP); T1095A is a compound which has been published under this name by Tanabe Seiyaku Comp. Ltd. (JP).

SGLT-5 is a new member of the SGLT-family and its function has not previously been identified. In this study SGLT-5 was cloned and a stable tetracycline-inducible T-Rex 293-hSGLT-5 cell line generated that was used for a first characterization of SGLT-5. Human SGLT-5 was identified as a mannose transporter but is also able to transport fructose to a lesser extent and AMG to a much lesser extent. Similarly to SGLT-2, SGLT-5 is predominantly expressed in the human kidney (Leicht et al., 2006). Together, these findings indicate that SGLT-5 could be a component in the renal reabsorption of mannose and fructose. However, the physiological relevance of human SGLT-5 in mannose and/or fructose homeostasis is not clear.

### REFERENCE EXAMPLE

SGLT5 was cloned as described in Example 1. However, instead of using the inducible pT-Rex-Dest30 plasmid the sequence was inserted into the expression plasmid pcDNA3.1/V5-His for constitutive expression. Using lipofection HEK/293 cells (ATCC) were stably transfected. The expression plasmid contained resistance to geneticin as a selection marker. The cells were cultivated in DMEM with 10% FCS and 0.6 μg/ml geneticin for selecting transfected cells. Expression was tested by RT-PCR.

Cells showing strong expression by PCR were subcloned and propagated. For functional assays using the FLIPR assay between 10,000 and 30,000 cells/well were utilized. As a positive control KCl was used in the tests. According to the fluorescence signal induced by KCl a number of 30,000 cells/well was determined to provide the highest fluorescence signal. The cells were washed, loaded and incubated as described in Example 1. Thereafter cells were exposed to glucose, galactose, fructose, AMG or mannose in different concentrations and changes in fluorescence were recorded as described in Example 1. KCl was used as a positive control and assay buffer as negative control. Moreover, the inhibitor phlorizin was used in some assay samples. The strongest signal for depolarisation could be observed for galactose, followed by glucose. Overall, however, the results showed only weak positive signals (see also Leicht S., 2005, and Leicht et al., 2006).

Furthermore, cells were tested for uptake of C¹⁴-labeled galactose in an assay as described in Example 1. A positive signal was observed for galactose at a concentration of 0.1 mM, however, the overall signal intensity was very low and only amounted to approximately 3-fold the negative control (as expressed in terms of absolute cpm). In addition, a competitive assay to inhibit C¹⁴-labeled AMG uptake into the transfected HEK cells was performed. Galactose showed the highest inhibitory effect, whereas mannose showed the lowest effect amongst all saccharides tested (see also Leicht et al., 2006).

Overall these results seem to indicate that SGLT5 is a galactose cotransporter. However, further testing revealed that these results were unreliable and irreproducible. Most likely they represent false positive signals and do not reflect the true function of SGLT5 as evidenced in Example 1. For example, in bona fide co-transport much higher readouts would be expected in the uptake experiments, both in terms of absolute values (cpm) as well as compared to background values of the control. Apparently the stably transfected cells constitutively expressing SGLT5 were subjected to a negative selective force resulting in loss of protein function and/or loss of protein expression over time.

### REFERENCES

Aschenbach J. R. et al., (2009) J. Physiol. Biochem 65(3):251-266

Bissonnette P. et al., (1999) J. Physiol. 520(2): 359-371.

Fujimori Yet al., (2008) J Pharmacol Exp Ther. 327(1):268-76.

Gilbert E. R. et al., (2007) Poultry Science 86:1739-1753.

Jung (2002) FEBS Letters 529:73-77.

Leicht, S. (2005) Klonierung and funktionelle Charakterisierung neuer SGLT-Gene, Diploma work submitted 2005 at Universitat Hohenheim, Germany

Leicht, S. et al., (2006) Diabetes; 55(suppl 1): 1512-P.

Pajor A M, (1994) Biochim Biophys Acta, 349-351.

Tazawa, S. et al., (2005) Life Sci. 76, 1039-1050.

Turk et al., (1996) J. Biol. Chem. 271: 1925-1934.

Turk&Wright (1996) J. Membrane Biol. 159, 1-20

Wright & Turk (2004) Europ. J. Physiol. 447: 510-518

Zhao F. Q. et al., (2005a) Am Dairy Sci Assoc. 88:2738-2748

Zhao F. Q. et al., (2005b) J. Dairy Sci. 88, 182-194.

1. A nucleic acid suitable for inducible expression of a member of the SGLT co-transporter family having the function of sodium dependent co-transport of mannose and/or fructose.

2. The nucleic acid according to claim 1, wherein the member of the SGLT co-transporter family is SGLT5, preferably human SGLT5.

3. The nucleic acid according to claim 1 selected from:
a) a nucleic acid encoding the amino acid sequence according to SEQ ID No. 2;
b) a nucleic acid according to SEQ ID No. 1;
c) a nucleic acid having at least 65% identity to a nucleic acid according to a) or b) and encoding a member of the SGLT co-transporter family having the function of sodium dependent co-transport of mannose and/or fructose; or
d) a nucleic acid sequence hybridizing under stringent conditions to the complement of any one of nucleic acid sequences according to a) to c) and encoding a member of the SGLT co-transporter family having the function of sodium dependent co-transport of mannose and/or fructose.

4. The nucleic acid according to claim 1, which is selected from a vector, plasmid, YAC or BAC.

5. The nucleic acid according to claim 1, which comprises an inducible expression system.

6. The nucleic acid according to claim 5, wherein the inducible expression system is characterized by an inducer selected from:
a) Tetracyline or tetracycline derivative doxycycline (Tet-on or Tet-off),
b) Ecdyson or muristerone A,
c) Mifepristone,
d) temperature shift acting on a temperature sensitive promoter,
e) transient expression by adenovirus mediated gene transfer, or
f) inducible expression by retrovirus mediated gene transfer.

7. A host cell comprising a nucleic acid according to claim 1.

8. The host cell according to claim 7, wherein the nucleic acid is integrated into a host cell chromosome or is located extrachromosomally.

9. The host cell according to claim 7 having the accession number DSM ACC3096.

10. A method of preparing cells inducibly expressing a member of the SGLT co-transporter family having the function of sodium dependent co-transport of mannose and/or fructose comprising the use of a nucleic acid according to claim

11. A method of screening for a compound capable of interacting with a protein encoded by a nucleic acid according to claim 1.

12. The method according to claim 11 comprising the use of a host cell comprising said nucleic acid.

13. The method according to claim 11 comprising the steps of:
a) contacting a cell expressing a protein encoded by said nucleic acid with one or more compounds; and
b) assessing the transport of a compound, which may be the same or different to the one or more compounds according to step a).

14. The method according to claim 11, wherein the transport and/or function is sodium dependent co-transport.

15. The method according to claim 11, wherein the compound is a small molecular weight chemical entity, preferably with a molecular weight below 500 Da, more preferred between 50 and 250 Da.

16. The method according to claim 11, wherein the compound is a sugar, optionally modified by the substitution of a hydroxylgroup by one or more of the chemical groups selected from —H (desoxy), -methyl, -ethyl, -propyl, -isopropyl, -oxomethyl, -oxoethyl, -oxopropyl, -oxoisopropyl, -acetyl, -amino, -imino and/or -acetamido.

17. The method according to claim 11, wherein the compound is a monosaccharide or a disaccharide.

18. The method according to claim 11, wherein the compound is one or more selected from glucose, fructose, mannose, galactose or alpha-methyl glucopyranoside.

19. The method according to claim 11, wherein the sugar is mannose and/or fructose.

20. The method according to claim 11, which is a competitive method.

21. A method of assessing the transport of a compound comprising the use of a nucleic acid according to claim 1 and/or a host cell comprising a nucleic acid according to claim 1.

22. A method for assessing the function of a compound interacting with a protein encoded by a nucleic acid according to claim 1.

23. An assay system comprising one or more selected from a nucleic acid according to claim 1, a SGLT co-transporter encoded by a nucleic acid according to claim 1, a host cell comprising a nucleic acid according to claim 1.

24. A kit comprising one or more selected from a nucleic acid according to claim 1, a SGLT co-transporter encoded by a nucleic acid according to claim 1, a host cell comprising a nucleic acid according to claim 1.

25. An article of manufacture comprising a nucleic acid according to claim 1, a SGLT co-transporter encoded by a nucleic acid according to claim 1, host cell comprising a nucleic acid according to claim 1.


SEQUENCE LISTING (9 sequences)

SEQ ID NO: 1 - DNA, 1791 bp, Homo sapiens
  CDS (1)..(1788)
atggccgccaactccaccagcgacctccacactcccgggacgcagctgagcgtggctgacatcatcgtcatcactgtgtattttgctctgaatgtggccgtgggcatatggtcctcttgtcgggccagtaggaacacggtgaatggctacttcctggcaggccgggacatgacgtggtggccgattggagcctccctcttcgccagcagcgagggctctggcctcttcattggactggcgggctcaggcgcggcaggaggtctggccgtggcaggcttcgagtggaatgccacgtacgtgctgctggcactggcatgggtgttcgtgcccatctacatctcctcagagatcgtcaccttacctgagtacattcagaagcgctacgggggccagcggatccgcatgtacctgtctgtcctgtccctgctactgtctgtcttcaccaagatatcgctggacctgtacgcgggggctctgtttgtgcacatctgcctgggctggaacttctacctctccaccatcctcacgctcggcatcacagccctgtacaccatcgcagggggcctggctgctgtaatctacacggacgccctgcagacgctcatcatggtggtgggggctgtcatcctgacaatcaaagcttttgaccagatcggtggttacgggcagctggaggcagcctacgcccaggccattccctccaggaccattgccaacaccacctgccacctgccacgtacagacgccatgcacatgtttcgagacccccacacaggggacctgccgtggaccgggatgacctttggcctgaccatcatggccacctggtactggtgcaccgaccaggtcatcgtgcagcgatcactgtcagcccgggacctgaaccatgccaaggcgggctccatcctggccagctacctcaagatgctccccatgggcctgatcatcatgccgggcatgatcagccgcgcattgttcccagatgatgtgggctgcgtggtgccgtccgagtgcctgcgggcctgcggggccgaggtcggctgctccaacatcgcctaccccaagctggtcatggaactgatgcccatcggtctgcgggggctgatgatcgcagtgatgctggcggcgctcatgtcgtcgctgacctccatcttcaacagcagcagcaccctcttcactatggacatctggaggcggctgcgtccccgctccggcgagcgggagctcctgctggtgggacggctggtcatagtggcactcatcggcgtgagtgtggcctggatccccgtcctgcaggactccaacagcgggcaactcttcatctacatgcagtcagtgaccagctccctggccccaccagtgactgcagtctttgtcctgggcgtcttctggcgacgtgccaacgagcagggggccttctggggcctgatagcagggctggtggtgggggccacgaggctggtcctggaattcctgaacccagccccaccgtgcggagagccagacacgcggccagccgtcctggggagcatccactacctgcacttcgctgtcgccctctttgcactcagtggtgctgttgtggtggctggaagcctgctgaccccacccccacagagtgtccagattgagaaccttacctggtggaccctggctcaggatgtgcccttgggaactaaagcaggtgatggccaaacaccccagaaacacgccttctgggcccgtgtctgtggcttcaatgccatcctcctcatgtgtgtcaacatattcttttatgcctacttcgcctga

SEQ ID NO: 2 - protein, 596 aa, Homo sapiens
MAANSTSDLHTPGTQLSVADIIVITVYFALNVAVGIWSSCRASRNTVNGYFLAGRDMTWWPIGASLFASSEGSGLFIGLAGSGAAGGLAVAGFEWNATYVLLALAWVFVPIYISSEIVTLPEYIQKRYGGQRIRMYLSVLSLLLSVFTKISLDLYAGALFVHICLGWNFYLSTILTLGITALYTIAGGLAAVIYTDALQTLIMVVGAVILTIKAFDQIGGYGQLEAAYAQAIPSRTIANTTCHLPRTDAMHMFRDPHTGDLPWTGMTFGLTIMATWYWCTDQVIVQRSLSARDLNHAKAGSILASYLKMLPMGLIIMPGMISRALFPDDVGCVVPSECLRACGAEVGCSNIAYPKLVMELMPIGLRGLMIAVMLAALMSSLTSIFNSSSTLFTMDIWRRLRPRSGERELLLVGRLVIVALIGVSVAWIPVLQDSNSGQLFIYMQSVTSSLAPPVTAVFVLGVFWRRANEQGAFWGLIAGLVVGATRLVLEFLNPAPPCGEPDTRPAVLGSIHYLHFAVALFALSGAVVVAGSLLTPPPQSVQIENLTWWTLAQDVPLGTKAGDGQTPQKHAFWARVCGFNAILLMCVNIFFYAYFA

SEQ ID NO: 3 - DNA, 2091 bp, Homo sapiens
agtccctcgggctcatacctagtgcctgcggcaggacagccatggccgccaactccaccagcgacctccacactcccgggacgcagctgagcgtggctgacatcatcgtcatcactgtgtattttgctctgaatgtggccgtgggcatatggtcctcttgtcgggccagtaggaacacggtgaatggctacttcctggcaggccgggacatgacgtggtggccgattggagcctccctcttcgccagcagcgagggctctggcctcttcattggactggcgggctcaggcgcggcaggaggtctggccgtggcaggcttcgagtggaatgccacgtacgtgctgctggcactggcatgggtgttcgtgcccatctacatctcctcagagatcgtcaccttacctgagtacattcagaagcgctacgggggccagcggatccgcatgtacctgtctgtcctgtccctgctactgtctgtcttcaccaagatatcgctggacctgtacgcgggggctctgtttgtgcacatctgcctgggctggaacttctacctctccaccatcctcacgctcggcatcacagccctgtacaccatcgcagggggcctggctgctgtaatctacacggacgccctgcagacgctcatcatggtggtgggggctgtcatcctgacaatcaaagcttttgaccagatcggtggttacgggcagctggaggcagcctacgcccaggccattccctccaggaccattgccaacaccacctgccacctgccacgtacagacgccatgcacatgtttcgagacccccacacaggggacctgccgtggaccgggatgacctttggcctgaccatcatggccacctggtactggtgcaccgaccaggtcatcgtgcagcgatcactgtcagcccgggacctgaaccatgccaaggcgggctccatcctggccagctacctcaagatgctccccatgggcctgatcatcatgccgggcatgatcagccgcgcattgttcccagatgatgtgggctgcgtggtgccgtccgagtgcctgcgggcctgcggggccgaggtcggctgctccaacatcgcctaccccaagctggtcatggaactgatgcccatcggtctgcgggggctgatgatcgcagtgatgctggcggcgctcatgtcgtcgctgacctccatcttcaacagcagcagcaccctcttcactatggacatctggaggcggctgcgtccccgctccggcgagcgggagctcctgctggtgggacggctggtcatagtggcactcatcggcgtgagtgtggcctggatccccgtcctgcaggactccaacagcgggcaactcttcatctacatgcagtcagtgaccagctccctggccccaccagtgactgcagtctttgtcctgggcgtcttctggcgacgtgccaacgagcagggggccttctggggcctgatagcagggctggtggtgggggccacgaggctggtcctggaattcctgaacccagccccaccgtgcggagagccagacacgcggccagccgtcctggggagcatccactacctgcacttcgctgtcgccctctttgcactcagtggtgctgttgtggtggctggaagcctgctgaccccacccccacagagtgtccagattgagaaccttacctggtggaccctggctcaggatgtgcccttgggaactaaagcaggtgatggccaaacaccccagaaacacgccttctgggcccgtgtctgtggcttcaatgccatcctcctcatgtgtgtcaacatattcttttatgcctacttcgcctgacactgccatcctggacagaaaggcaggagctctgagtcctcaggtccacccatttccctcatggggatcccgaggccccaagaggggcagattcccctcacagctgcacagcagctcggtgcccaagaactggccaagccagcaaagcgggagccctgaaaaattaggggggaaatgggagaaaataatgtgacatttcaaaaacagcaccaaagcagtcagcattggaaggaaaattagatttctgacggacatcctg

SEQ ID NO: 4 - DNA, 23 bp, artificial
  PCR primer
caccgccatggccgccaactcca

SEQ ID NO: 5 - DNA, 33 bp, artificial
  PCR primer
tcaggcgaagtaggcataaaagaatatgttgac

SEQ ID NO: 6 - DNA, 21 bp, artificial
  PCR primer
gatgacctttggcctgaccat

SEQ ID NO: 7 - DNA, 19 bp, artificial
  PCR primer
gtcccgggctgacagtgat

SEQ ID NO: 8 - DNA, 18 bp, artificial
  PCR primer
tggcatggactgtggtca

SEQ ID NO: 9 - DNA, 18 bp, artificial
  PCR primer
gcaccaccaactgcttag